(19) United States
(12) Patent Application Publication
(10) Pub. No.: US 2003/0216752 A1
(43) Pub. Date: Nov. 20, 2003

(54) Device for laparoscopic tubal ligation

(75) Inventors: Warren P. Williamson IV, Loveland, OH (US); Craig B. Berky, Milford, OH (US); Thomas J. Ward, Columbus, OH (US); George Keller, Columbus, OH (US); Timothy N. Wells, Ridgefield, CT (US); William J. Christy, Oviedo, FL (US)

Correspondence Address: Kevin G. Rooney, Wood, Herron & Evans, L.L.P., 2700 Carew Tower, 441 Vine Street, Cincinnati, OH, 45202-2917, US

(73) Assignee: ESD Medical L.L.C.

(21) Appl. No.: 10/435,823

(22) Filed: May 12, 2003

Related U.S. Application Data
(63) Continuation of application No. PCT/US01/43598, filed on Nov. 14, 2001.
(60) Provisional application No. 60/248,463, filed on Nov. 14, 2000.

(51) Int. Cl. A61B 17/04

(52) U.S. Cl. 606/139; 606/148

(57) ABSTRACT

The present disclosure relates to a ligating instrument and end effector assemblies for use in laparoscopic tubal ligation procedures. The ligating instrument includes an integral grasping assembly and an integral end effector actuator and is configured to perform a ligating procedure by operation of a single hand of a user. The ligating instrument includes an integral grasper assembly for grasping a tubular tissue section and removable end effector or suture deployment mechanism for holding open a suture to be positioned about the tubular section. The grasper is configured to draw the tubular section into the open loop of suture material and cinch the suture material about the tubular tissue section. A rotator knob may be provided to orient grasper members associated with the grasper assembly relative to the tissue section. An alternative end effector is provided to removably mount on the distal end of the ligating instrument and to cut that part of the tubular tissue section which needs to be removed after the tubular tissue section has been ligated. A relatively soft over mold section may be provided to at least partially enclose the handle of the ligating instrument in order to provide comfort and facilitate a surer grip of the instrument.

### CROSS REFERENCE TO RELATED APPLICATIONS

The present disclosure claims priority to U.S. Provisional Application Serial No. 60/248,436, filed Nov. 14, 2000, and entitled, “Device for Laparoscopic Tubal Ligation”.

### BACKGROUND

1. Field of the Invention

The present invention relates to surgical devices and methods and, more particularly, to devices and methods for grasping a tissue segment and delivering a loop of suture material to the tissue segment with a single, one hand operable ligating instrument for the purpose of ligation.

2. Description of Related Art

Modern surgical techniques often entail the use of endosurgery, wherein large incisions are avoided, and, instead, elongated instruments are inserted into and manipulated through trocars. Typically the surgical site, such as the peritoneum, is viewed remotely, and the surgeon works while watching a monitor.

Endoscopic applications of preknotted suture loops include the ligation of protruding pieces of tissue such as polyps or ends of blood vessels. Several devices are known in the art that deliver such suture loops to a site generally remote from the surgeon's hand and are remotely manipulable.

The tonsillotome of Longino (U.S. Pat. No. 1,468,599) contains a shank having a lumen wherein a suture loop resides and from which the loop is manipulable around a piece of tissue. Previously disclosed devices include those of Neivert (U.S. Pat. No. 1,833,687), who discloses a surgical snare comprising a fixed and a movable member capable of relative movement for tightening the snare loop that has been housed in the bore of the movable member.

The ligating loop device of Bueina (U.S. Pat. No. 5,242,459) has a shaft with a suture loop at its distal end, the loop having a slip knot. In addition, the device has a cutting element for severing the loop once it has been tightened. As with the device of Neivert, relative movement between two cylindrical members causes a tightening of the knot.

Noda et al. (U.S. Pat. No. 5,320,629) also contains relatively slidable members, one of which carries a suture with a slip knot at the end.

The endoscopic loop applying instrument of Kinet et al. (U.S. Pat. No. 5,405,351) both delivers the loop and cuts the free end of suture material.

The ligating instrument of Yoon (U.S. Pat. No. 5,486,186) also delivers a loop of suture material, the material extending through the lumen of an elongated device for being operated upon to tighten the loop around a piece of tissue. A cutter is also disclosed that is positioned within the lumen and is externally operable.

An exemplary device for ligating tissue is disclosed in U.S. Pat. No. 6,152,936 to Christy et al.(the entire disclosure of which is incorporated by reference herein) which discloses a novel suture management device including legs to hold a loop of suture material open to position around tissue.

A problem shared by the above devices is the need for multiple instruments and, more importantly, the need to use two hands to operate the instruments to grasp and ligate the tissue structure.

### SUMMARY

There is disclosed a ligating instrument configured for single handed operation which includes integral grasping assembly to grasp a tissue section along with suture management assembly to hold open a loop of suture material such that the grasped tissue can be drawn through the material. The ligating instrument generally includes a handle assembly, an elongate tubular member extending distally from the handle assembly and an end effector mounted on a distal end of the elongate tubular member and operable to hold open a loop of material. A grasping member is extendable from a distal end of the elongate tubular member and a grasp actuator, integral with the handle assembly is provided such that actuation of the grasp actuator moves the grasping member relative to the distal end of the elongate tubular member.

The grasper assembly includes a grasper pusher which is configured to extend a pair of flexible legs beyond the distal end of an elongated tubular member of the ligating instrument in order to grasp tissue. Springs are provided to bias the grasper members in a retracted position within the elongated tubular member. Preferably a thumbwheel is provided at the proximal end of the instrument to rotate and orient the grasper legs relative to the tissue to be grasped.

The above described interval suture ligating mechanism including a lever configured to draw a suture held by an end effector about a tubular tissue section and cut the suture. A first end effector is provided for removable mounting on a distal end of the elongate tubular member of the ligating instrument and generally includes pivotal legs having fingers at their distal end for holding a section of suture in an open loop configuration. Preferably the legs have ramps formed on them such that upon advancement of the grasper members the legs are cammed open to hold the suture in an open condition. A cutter may be provided on the first end effector to cut the length of suture material upon actuation of the device.

A second end effector is provided and is removably mountable on the distal end of the elongate tubular member. The second end effector is configured such that upon actuation of the ligating instrument a continuous loop of suture material draws the tubular tissue section to be excised against a cutting blade associated with the second end effector. Preferably, the second end effector includes a single arm having a similar ramp which engages the grasper and maintains the loop in an open configuration.

A resilient or flexible material may be formed as an over mold over at least a portion of the handle of the ligating instrument in order to provide comfort to the user and ensure a firmer grip.

### DESCRIPTION OF THE DRAWINGS

Various embodiments are described herein with reference to the drawings wherein:

FIG. 1 is a perspective view of one embodiment of the disclosed ligating instrument with half of the handle body removed;

FIG. 2 is a perspective view of a first handle body half;

FIG. 3 is a perspective view of a second handle body half;

FIG. 4 is a perspective view of the assembled first and second handle body halves;

FIG. 5 is a perspective view of a handle subassembly of a grasper assembly;

FIG. 6 is a sectional view of the handle assembly;

FIG. 7 is a partial enlarged sectional view showing the area of detail A of FIG. 6;

FIG. 8 is a partial enlarged sectional view showing the area of detail B of FIG. 6;

FIG. 9 is a perspective view of a grasper subassembly of the grasp assembly;

FIG. 10 is a perspective view of a handle subassembly of the handle assembly;

FIG. 11 is a sectional view of the distal end of the ligating instrument;

FIG. 12 is a side view of a cutter tube;

FIG. 13 is a perspective view of the cutter tube;

FIG. 14 is a side view of a pull tube;

FIG. 15 is a perspective view of the pull tube;

FIG. 16 is a side view, partly shown in section, of a grasp tube;

FIG. 17 is a perspective view of an outer tube;

FIG. 18 is a perspective view of a ligation squid assembly;

FIG. 19 is a side view, in section, of a stiff deflecting leg;

FIG. 20 is a side view of a pinned leg;

FIG. 21 is a side view of a middle leg;

FIG. 22 is a perspective view of the cutting blade of the ligation squid assembly;

FIG. 23 is a perspective view of the suture spool of the ligation squid assembly;

FIGS. 24 and 25 are perspective views of an alternative embodiment of a suture management squid device for use with a ligation squid assembly similar to that shown in FIG. 18;

FIG. 26 is a perspective view of a transecting squid assembly for use with the disclosed ligating instrument;

FIGS. 27 and 28 are perspective views of the squid body of FIG. 26;

FIG. 29 is a perspective view, with parts separated, of the spool assembly of FIG. 26; and

FIGS. 30-32 are perspective views of an over molded handle for use with the disclosed ligating instrument.

### DETAILED DESCRIPTION OF PREFERRED EMBODIMENTS

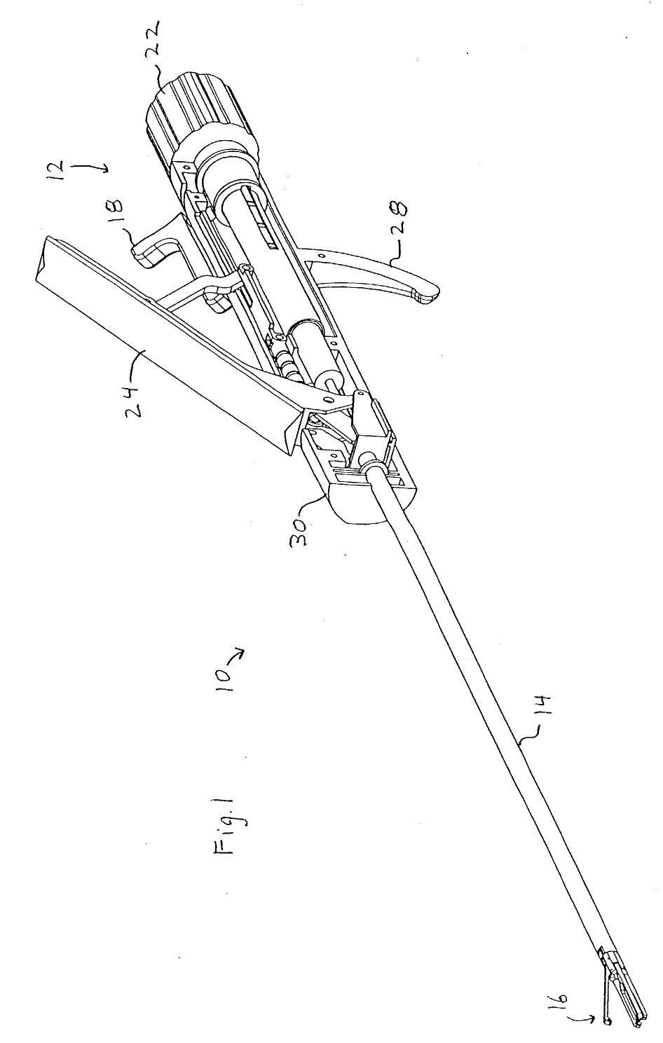

Referring to FIG. 1, a ligating instrument is shown which is configured to grasp and ligate a tubular structure with a single instrument while being operated by a single hand of the user. The disclosed ligating instrument 10 generally includes a handle assembly 12 and an elongated outer tube 14 extending distally from handle assembly 12. Various removable end assemblies or single use loading units “SLLU's” 16 are removably mounted on a distal end of outer tube 14. As used herein, the term “proximal” means the end of a component or part thereof closer to the operator while the term “distal” refers to that component or part thereof further from the operator. A grasper pusher 18 is movably mounted with respect to handle assembly 12 and functions along with a grasp subassembly 20 to extend and retract tissue grasp legs (described below) from the distal end of outer tube 14. A handle thumbwheel 22 is rotatably mounted on a proximal end of handle assembly 12 and 13 provided to rotate tissue grasp legs relative to outer tube 14 to orient the grasp legs relative to tissue. A handle lever 24 is pivotably mounted to handle assembly 12 and functions with a handle subassembly 26 to actuate various SULU's 16. Preferably, ligating instrument 10 is formed of steel, however, other biocompatible materials such as titanium, metals, plastics or ceramics can be used.

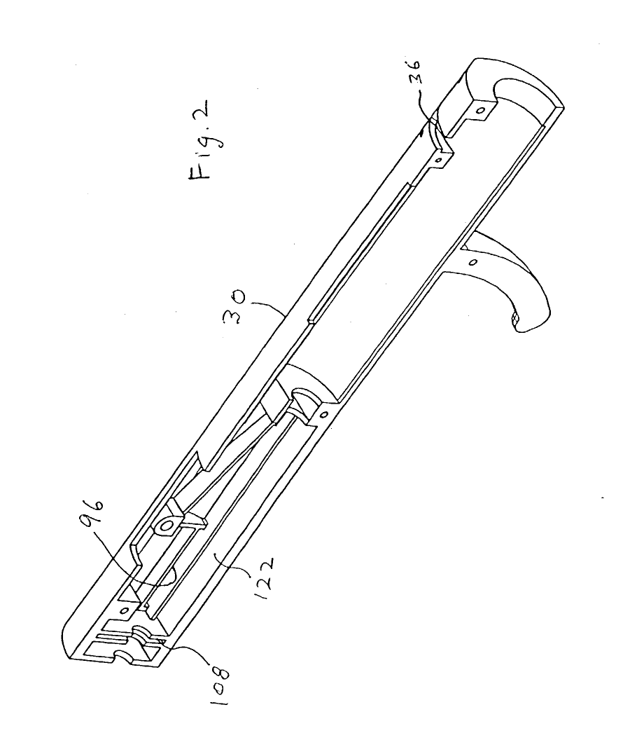

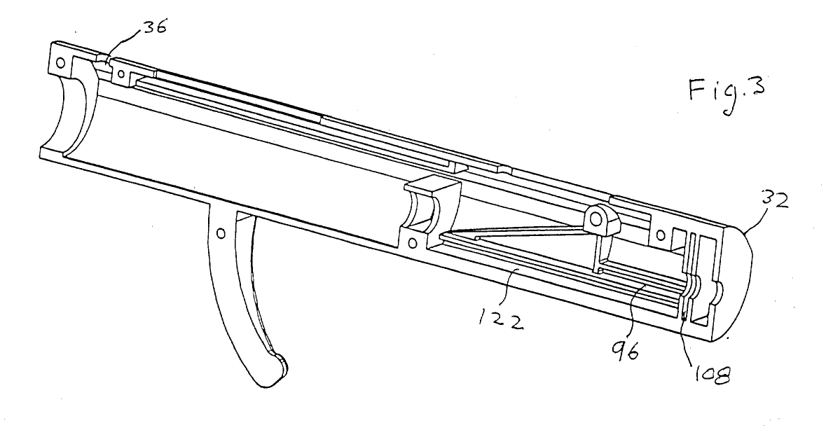

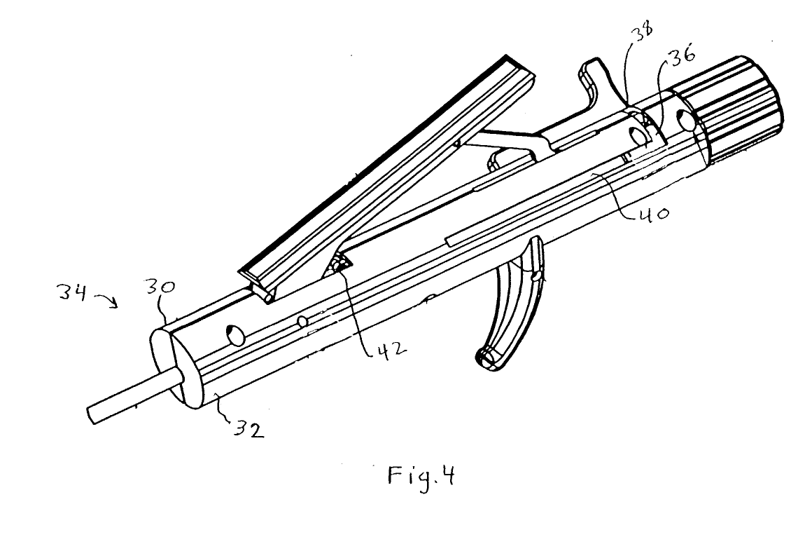

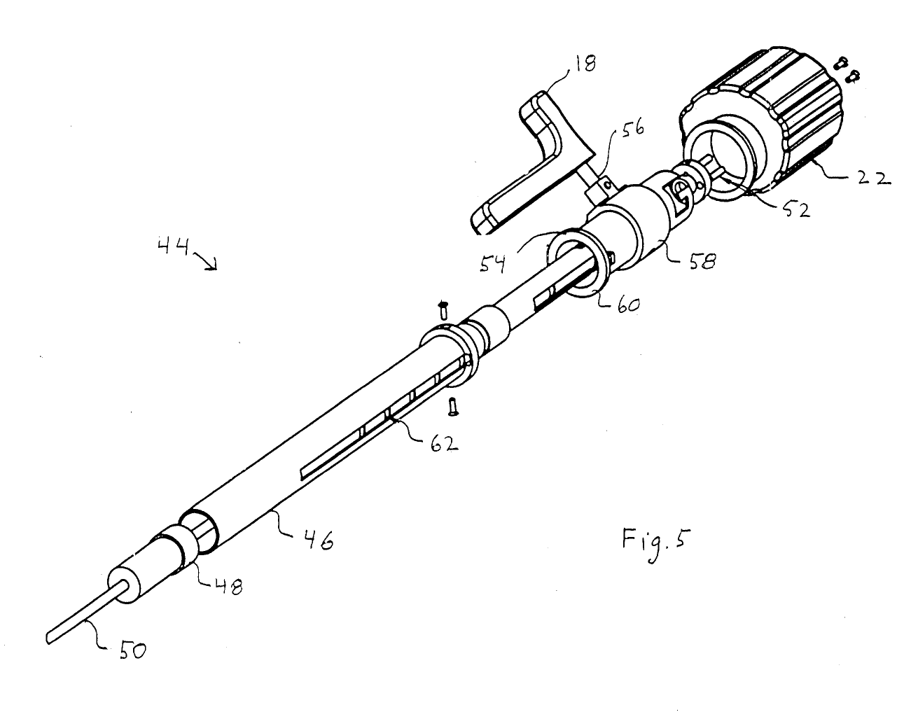

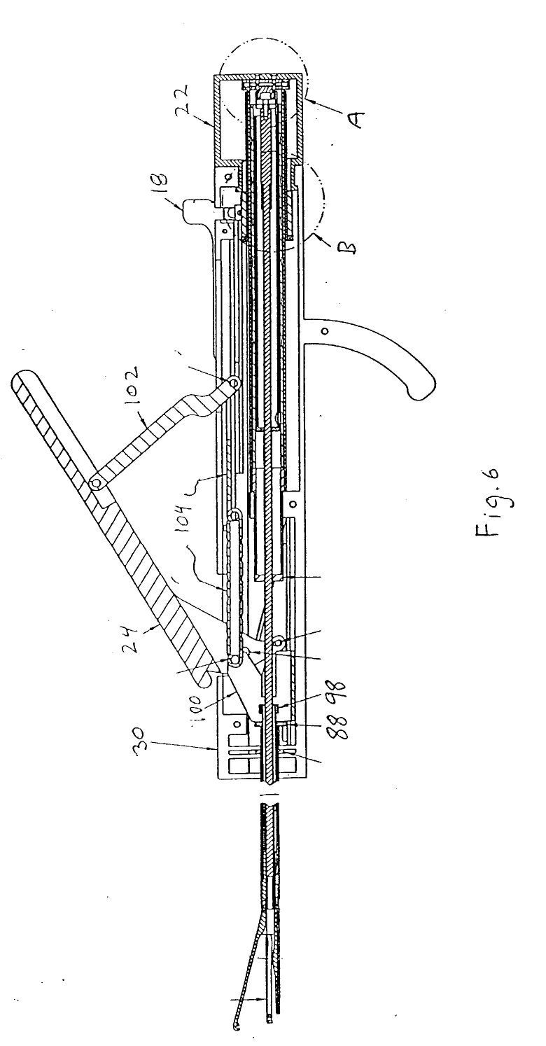

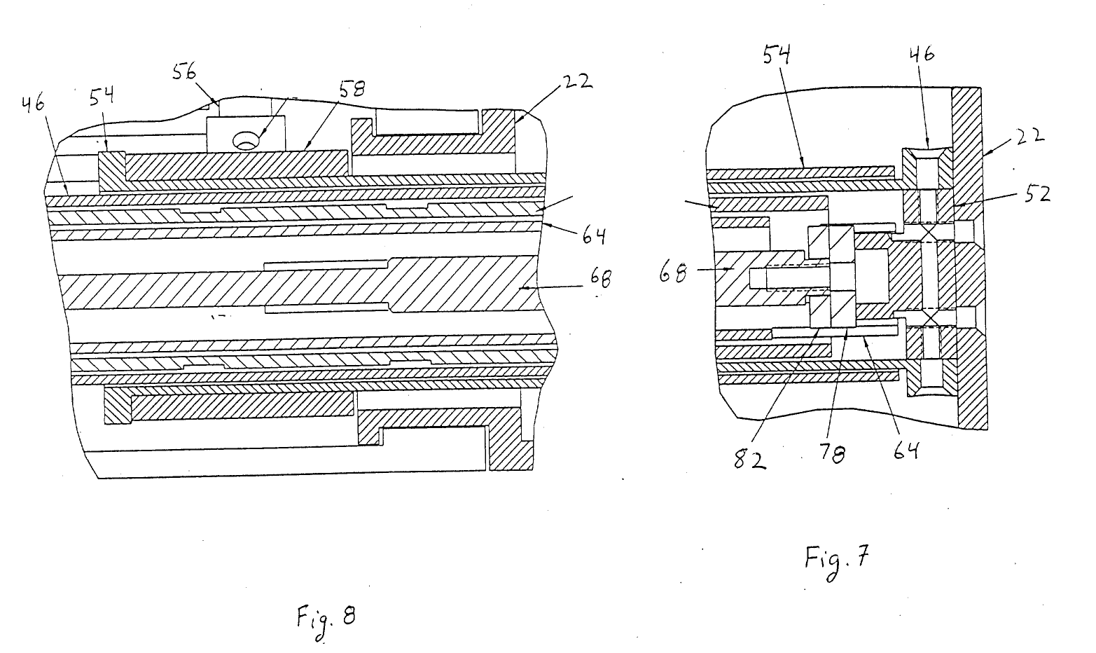

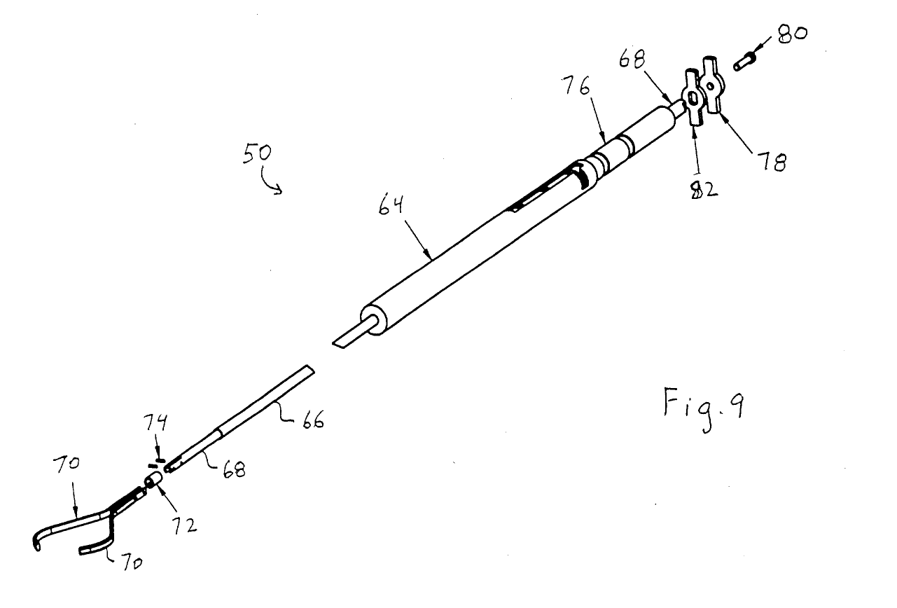

Ligating instrument 10 is particularly designed to be used in a single-handed fashion and includes an ergonomically curved depending leg 28 to facilitate one-handed use of ligating instrument 10. Preferably, handle assembly 12 includes a first handle body half 30 (FIG. 2) and a second handle body half 32 (FIG. 3). First and second handle body halves 30 and 32, respectively, are mirror images, i.e. complimentary components which combine to form a handle body 34 (FIG. 4). Referring for the moment to FIGS. 2, 3 and 12, outer tube 14 includes a proximal flange 106 which fits in channels 108 in first and second handle body halves 30, 32. Outer tube 14 also has a bayonet type slot 110 at the distal end for receipt of various end assemblies.

As shown in FIGS. 2-4, handle body 34 defines a transverse pusher slot 36 and a pair of longitudinal pusher slots 38 and 40 radially spaced apart and extending distally from transverse pusher slot 36. By moving grasper pusher 18 through transverse pusher slot 36, grasper pusher can be aligned with either of longitudinal pusher slots 38 or 40 to facilitate right or left handed use. Handle body 34 also defines an opening 42 for handle lever 24.

Referring now to FIGS. 5-9, grasp subassembly 20 will now be described. Referring initially to FIG. 5, a grasp handle subassembly 44 of grasper assembly 20 includes a grasp spring tube 46 having a rotatable plug 48 at a distal end of spring tube 46. A grasper subassembly 50 of grasper assembly 20 extends through grasp handle subassembly 44 and extends distally therefrom through outer tube 14. A grasper plug 52 is provided at a proximal end of grasp handle subassembly 44 to connect thumbwheel 22 to grasp subassembly 20 and thereby allow grasp subassembly 20 to rotate about the longitudinal axis of ligating instrument 10 in order to orient grasper legs with respect to the tissue to be ligated.

Grasp handle subassembly 44 further includes a grasp collar 54 which is longitudinally slidable over grasp spring tube 46.

As shown, grasper pusher 18 includes a thumbshaft 56 which is attached to a grasp clevis 58. Grasp clevis 58 is rotatably mounted about grasp collar 54 so as to allow grasper pusher 18 and thumbshaft 56 to move through transverse pusher slot 36 in handle body 34. This allows switching from right to left hand operation and visa versa. A distal edge of grasp clevis 58 abuts a flange 60 on grasp collar 54 to drive grasp collar 54 longitudinally in response to longitudinal movement of grasper pusher 18 and thumbshaft 56 through one of longitudinal pusher slots 38 or 40 in handle body 34.

Grasp handle subassembly 44 also includes a spring 62 positioned inside spring tube 46 which biases grasp collar 54 and thus grasper pusher 18 proximally. Grasp collar 54 is connected to grasper subassembly 50 of grasper subassembly 20 as described below.

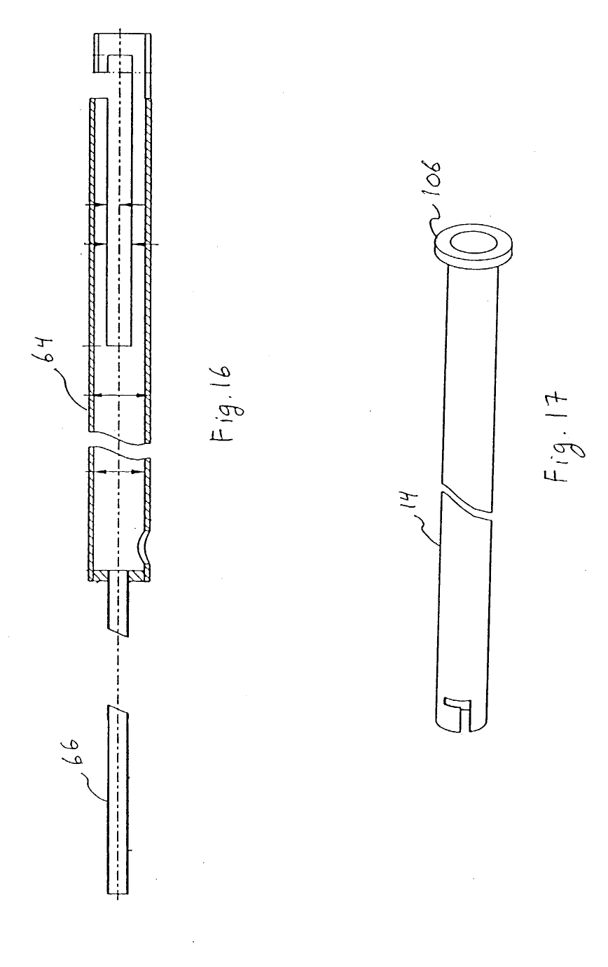

Referring now to FIG. 9, grasper subassembly 50 of grasp subassembly 20 will now be described. As noted above, grasper subassembly 50 extends through outer tube 14 and handle subassembly 44. A proximal end of grasper subassembly is connected to grasp collar 54. Grasper assembly 50 generally includes a grasp tube 64 movably mounted within grasp spring tube 46. A grasp tube extension 66 extends distally from grasp tube 46 and through outer tube 14 to a distal end thereof. (See also, FIG. 16)

A return spring driver assembly 68 extends through grasp tube 64 and grasp tube extension 66. A pair of flexible grasper legs 70 are mounted on a distal end of return spring driver assembly by means of an end piece 72 and pins 74. When return spring driver assembly is in a proximal position relative to handle body 34, grasper legs 70 are drawn into grasp tube extension 66 and assume a generally flat configuration. A second spring 76 is positioned within grasp tube 64.

A first driver plate 78 is affixed to a proximal end of return spring driver assembly 68 by means of a pin 80. A second driver plate 82 is movably mounted over driver assembly 68 and contacts second spring 76 and a proximal end of grasp tube 64.

In operation, a first movement or throw of grasper pusher 18 within one of longitudinal pusher slots 38, 40, drives grasper subassembly 50 distally within grasp spring tube 46 against the bias of first spring 62. This extends the distal end of grasp tube extension 66 out of the distal end of outer tube 14. Further distal movement of grasper pusher 18 moves return spring driver assembly 68 distally against the further bias of second spring 76 to thereby extend grasper legs 70 out of and beyond the distal end of grasp tube extension 66 and thus beyond the distal end of outer tube 14 and allows the legs 70 to spring open to grasp tissue.

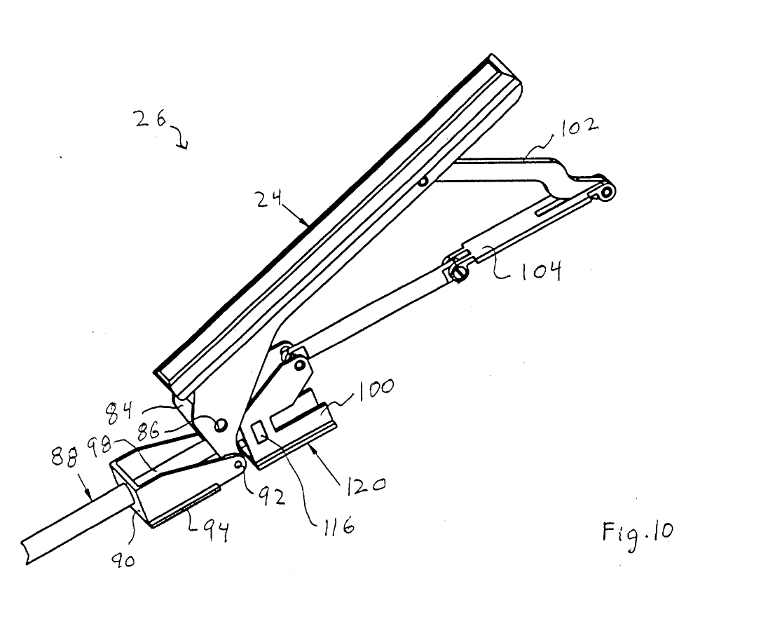

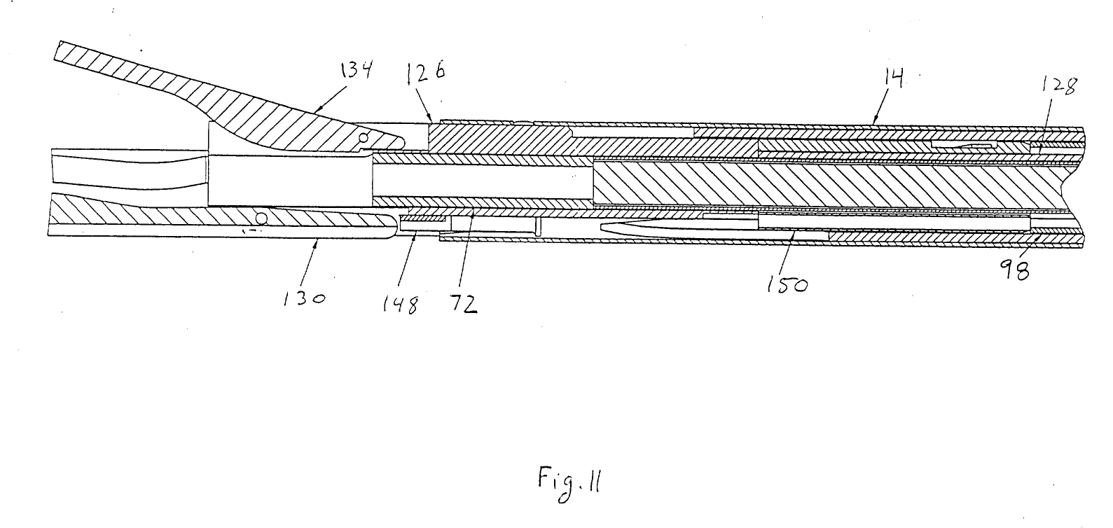

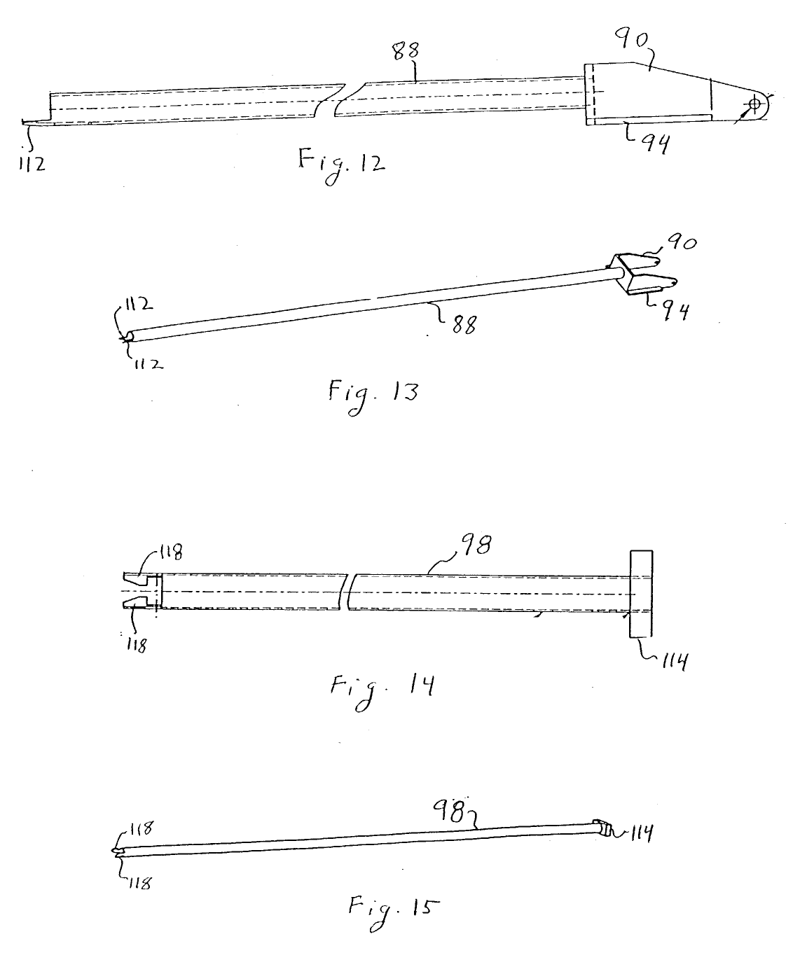

Referring now to FIGS. 6-8 and 10-11 there will now be described handle subassembly 26. Referring initially to FIG. 10, handle subassembly 26 includes handle lever 24, which has a drive bracket 84 pivotally mounted to handle body 34 by pins (not shown) which extend from first and second handle body halves 30 and 32 and into corresponding holes 86 in drive bracket 84. Handle subassembly 26 also includes a cutter tube 88 having a mounting bracket 90 at its proximal end. Mounting bracket 90 is pinned to drive bracket 84 by pins 92. Mounting bracket 90 includes a pair of lips 94 which are longitudinally guided by slots 96 in first and second handle body halves 30 and 32 (FIGS. 2 and 3). Thus, movement of handle lever 24 towards handle body 34 drives cutter tube 88 distally, while movement of handle lever 24 away from handle body 34 retracts cutter tube 88 in the proximal direction. Referring for the moment to FIGS. 12 and 13, cutter tube has projections 112 on the distal end to cam a cutting blade on an end assembly to cut suture material.

Handle subassembly 26 further includes a pull tube 98 having a fire clevis 100 affixed to a proximal end of pull tube 98. As shown in FIGS. 14 and 15, pull tube 98 has a flange 114 at its proximal end which held in notches 116 in fire clevis 100 (FIG. 10). Pull tube 98 has projections 118 at a distal end to engage a corresponding suture spool on an end assembly to cinch a suture. Referring to FIGS. 2, 3 and 10, fire clevis 100 has lips 120 which are longitudinally guided in slots 122 in first and second handle body halves 30, 32.

Handle lever 24 has a handle link 102 pivotally mounted at a first end near the proximal end of handle lever 24. A two part linkage 104 connects a second end of handle link 102 to fire clevis 100. When handle lever 24 is moved toward handle body 34, fire clevis 100, and thus pull tube 98, are drawn proximally within handle body 34. Similarly when handle lever 24 is pivoted away from handle body 34, pull tube 98 is moved distally within outer tube 14.

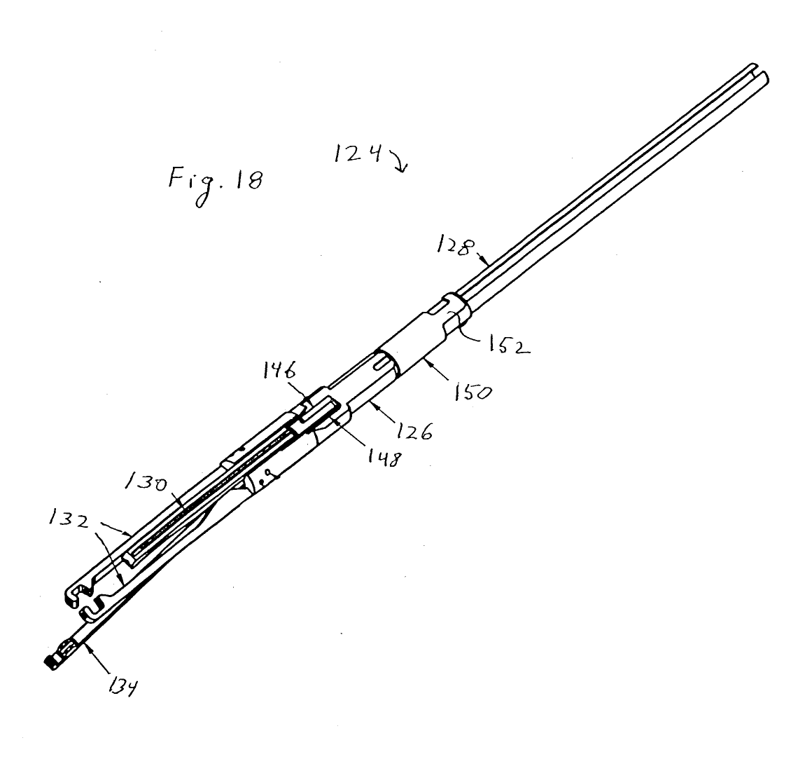

Referring now to FIG. 18, there is disclosed an end effector or SULU particularly adapted to be used with ligating instrument 10. Ligating squid assembly 124 generally includes a squid body 126 having a squid to 128 extending proximally from squid body 126. A plurality of suture supporting legs are pivotally mounted to squid body 126 and extend distally therefrom. Specifically, a stiff leg 130 extends distally from squid body 126 and is configured to retain a segment of suture material and a slip knot at the end of stiff leg 130. Squid body 126 also includes a pair of pinned legs 132 which are pivotally connected to squid body 126 and a middle leg 134 which is also pivotally connected to squid body 126. Referring for the moment to FIG. 19, stiff leg 130 generally includes a guide slot 138 running partially along the length thereof as well as a suture hole formed in the distal end of stiff leg 130. A length of suture (not shown) is threaded through suture hole 136 into guide slot 138 and the guide tube 140 connects guide slot 138 with suture hole 136. A partial ramp 140 is formed on the underside of stiff leg 130 to partially flex stiff leg 130 away from the center line of the instrument as grasper legs 70 are extended past stiff leg 130.

Referring to FIG. 20, there is illustrated one of a pair of pinned legs 132 which include fingers 142 at a distal end thereof to support a portion of a suture loop. Similarly, referring to FIG. 21, middle leg 134 also has fingers 142 to assist in maintaining an open suture loop.

Referring back to FIG. 14, ligation squid assembly 124 further includes a guide tube 146 in alignment with guide slot 138 of stiff leg 130 to receive a length of suture therethrough. A cutting blade 148 is positioned adjacent to guide tube 146 and is flexible in response to engagement with projections 112 of cutting tube 88 so as to cam the cutting blade into engagement with the suture and sever it.

A ligating squid assembly 124 further includes a suture spool 150 slidably mounted about squid body 126. Suture spool 150 is configured to retain a free end of a length of suture and, upon proximal movement of suture spool 150, cinch a suture around tissue positioned within the loop of suture held by the squid arms. Arms 152 extend from a proximal end of suture spool 150 and are configured to engage projections 118 on pull tube 98 such that when pull tube 98 is retracted proximally it draws suture spool 150 and thus the length of suture proximally. As shown in FIGS. 20 and 21, pinned legs 132 and middle leg 134 include ramps 154 and 156, respectively. Ramps 154 and 156 deflect legs 132 and middle leg 134 upon engagement with grasper legs 70 as grasper legs 70 are extended out the distal end of ligating instrument 10.

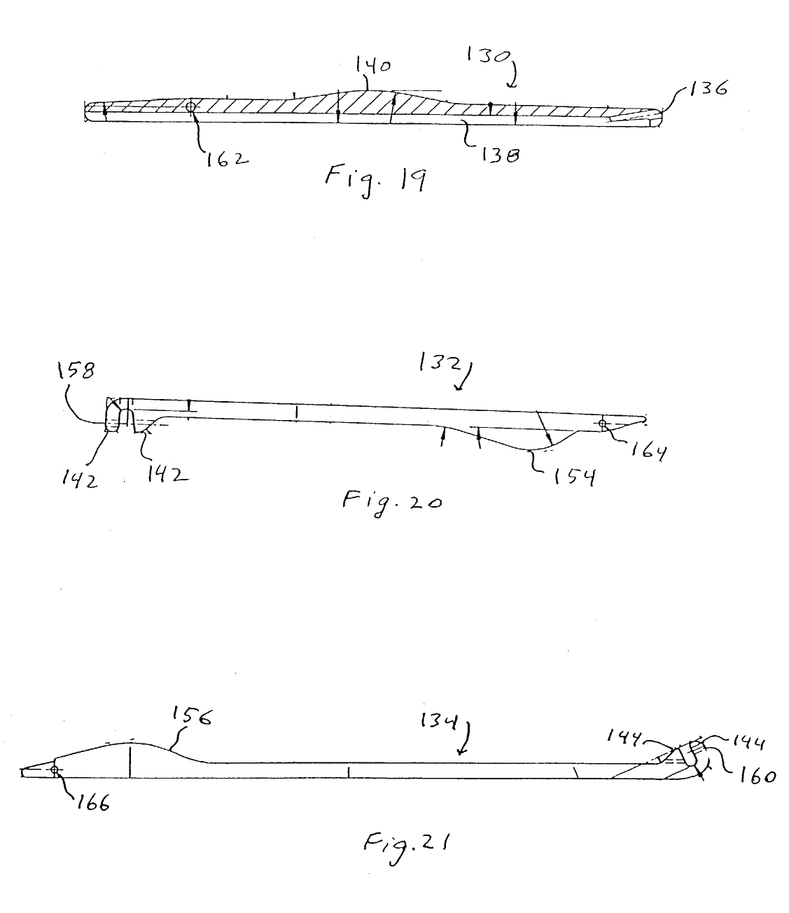

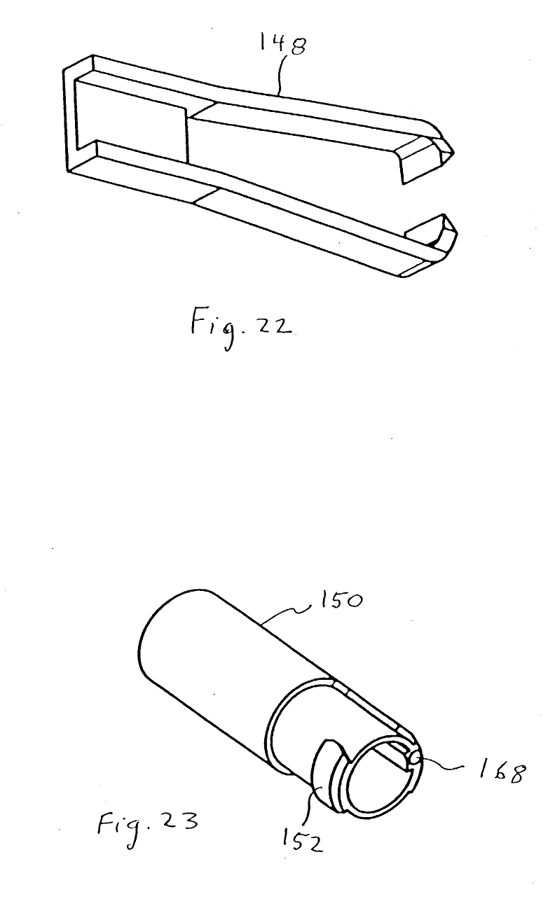

Referring now to FIGS. 24 and 25, there is disclosed an integrally molded squid device for use with a ligation squid assembly similar to that of ligation squid assembly 124. Molded squid device 170 is an integral structure, preferably formed of a plastic material. Squid device 170 generally includes a base 172 having a stiff leg 174 extending distally therefrom. Stiff leg 174 includes a channel 176 and a bore 178 for receipt of a length of suture. Squid device 170 further includes a pair of side legs 180 which have ramps 182 for engagement with a grasper device to bias legs 180 outwardly in a manner similar to that described hereinabove. Said legs 180 also have fingers 184 to releasably retain a section of suture and maintain it in an open loop configuration.

Molded squid device 170 further includes a bottom leg 186 having a ramp 188 for engagement with a grasper device. Bottom leg 186 also includes a fingers 190 to releasably retain a section of the suture material. Stiff leg 174, side legs 180 and bottom leg 186 are connected to base 174 by living hinges 192, 194, and 196, respectively. Base 172 also includes a socket 198 for mounting squid device to a modified ligating squid assembly.

It should be noted that by integrally molding squid device 170 as a unitary structure, squid device 170 takes advantage of the inherent flexibility characteristics of a living hinge to retain the legs in a generally collapsed configuration.

Referring now to FIG. 26, there is illustrated a unique transecting squid assembly for use with the disclosed ligating instrument 10 to cut the loop of tissue remaining after being ligated with the suture of ligating squid assembly 124. Transecting squid assembly 200 is configured to be reusable and releasably mountable in the distal end of outer tube 14. Transecting squid assembly 200 is provided to grasp and cut a previously ligated tissue section.

Transecting squid assembly 200 generally includes a squid body 202 having a middle leg 204 pivotally mounted on squid body 202 as shown. Middle leg 204 includes fingers 206 at its distal end to hold a portion of a continuous loop of wire in an open loop configuration. A squid tube 208 extends proximally from squid body 202 and is insertable within outer tube 14. Transection squid assembly 200 further includes a spool assembly 210 slidably mounted on squid tube 208 and configured to secure a portion of a the loop of material. A pair of cutting blades 212 extend from the distal most tip of squid body 202. Blades 212 are provided to cut anatomical tissue drawn against blades 212 by contraction of the closed loop of wire material.

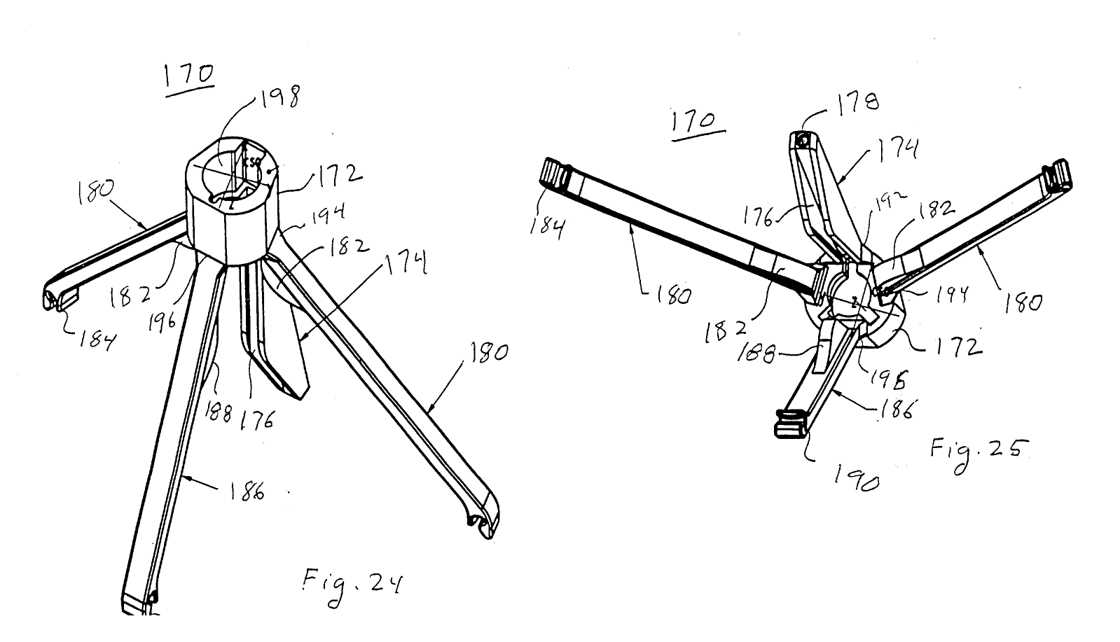

Referring for the moment to FIGS. 27 and 28, an underside of squid body 202 includes a longitudinally extending wire channel 214 which is configured to retain and guide a segment of a continuous wire loop.

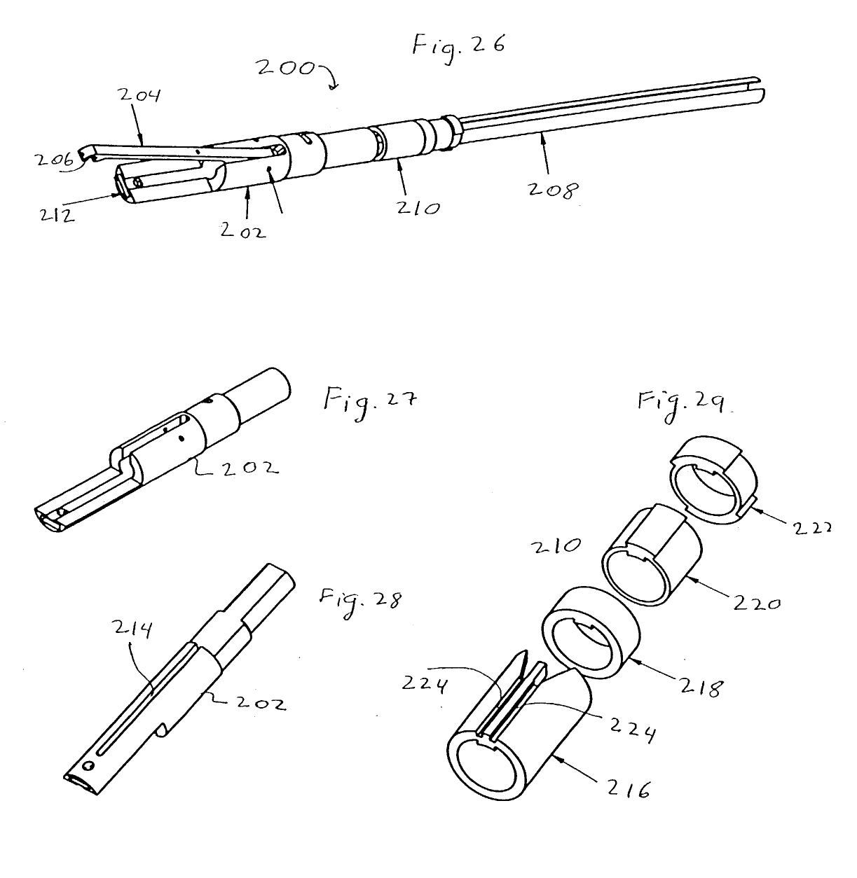

Referring now to FIG. 29, the spool assembly 210, which is slidably mounted on squid tube 208 generally includes four subsections 216, 218, 220 and 222 which are welded together. A portion of a closed wire loop is secured within channels 224 in part 216.

The above disclosed ligating instrument is particularly suitable for ligating anatomical tubular structures, such as, for example Fallopian tubes. The operation of ligating instrument 10 to ligate a Fallopian tube in the manner of an endoscopic, Pomeroy type procedure will now be described. Initially, a ligating squid assembly 124 is inserted and affixed to a distal end of ligating instrument 10. As noted above, ligating squid assembly 124 includes a bayonet type fitting which engages the distal end of outer tube 14. Prior to insertion in the body, the legs of ligation squid assembly 124 are in a collapsed configuration generally in alignment with the longitudinal axis of outer tube 14. Handle lever 24 is in an open position and grasp pusher 18 is in a proximal most position. As noted hereinabove, the instrument is specifically designed to be operated with a single hand, i.e., a one hand use instrument. In grasping the instrument, the pinky and ring finger of the operable hand rest upon depending leg 28 while the middle finger and index finger rest upon handle lever 24. The thumb is positioned adjacent grasp pusher 18. As further noted hereinabove, by initially moving grasper pusher 18 through transverse slot 36 in handle body 34, the ligating instrument 10 can be configured for case of use with either hand.

Ligating instrument 10 is then inserted into the body such that ligating squid assembly 124 is positioned adjacent a Fallopian tube. Thereafter, the thumb can be pushed against grasper pusher 18 driving grasper pusher 18 distally to initially extend grasp tube extension 66 towards the distal end of outer tube 14. Further pressure through a second throw or distal distance of grasper pusher 18 extends grasper legs 70 out the distal end of outer tube 14. As grasper legs 70 extend beyond the distal end of outer tube 14, grasper legs 70 engage ramps 154 and 156 on pinned legs 132 and 134 camming legs 132 and 134 into an open position. Grasper legs 70 extend beyond the ends of legs 132 and middle leg 134 and are positioned about the tubular structure. It should be noted that as grasper legs extend out the distal end of outer tube 14 they extend through the open suture loop held by stiff leg 130, pinned leg 132 and middle leg 134.

Upon slow release of grasper pusher 18 against the bias of the return springs, grasper legs 70 grasp the anatomical tubular structure and draw a tubular structure through the open loop of suture material. Once grasper pusher 18 is returned to a fully proximal most position the fingers may be operated to actuate handle lever 24. As handle lever 24 is moved toward handle body 34, pull tube 98, which is attached at its distal end to spool 150 draws spool 150 and thus the attached section of suture material proximally thereby contracting the loop of suture material about the Fallopian tube and pulling the suture material free of the associated legs. It should be noted that the proximal movement of pull tube 98 is a fairly long throw relative to the forward movement of cutter tube 88 which also happens in response to depression of lever 24. As cutter tube 88 is advanced it cams cutting blade 148 against the suture material at a point adjacent guide tube 146 to thereby sever the suture material. Grasper pusher 18 can then be advanced to release the now sutured and ligated Fallopian tube structure.

Ligating instrument 10 is then withdrawn from the body and ligating squid assembly 124 is removed from ligating instrument 10.

In order to cut the now ligated loop section of tubular tissue structure, transecting squid assembly 200 is assembled onto the distal end of ligating instrument 10 and ligating instrument 10 is reinserted into the body adjacent the ligated Fallopian tube. Advancement of grasper pusher 18 again advances grasper legs 70 towards the anatomical tubular structure camming open the middle squid leg 204 of transecting squid assembly 200. Grasper pusher 18 is again operated to regrasp the tissue with grasper leg 70 and draw it into the continuous wire loop of material supported by transacting squid assembly 200. Handle lever 24 is then again actuated to draw spool assembly 210 proximally thereby drawing the ligated loop of tissue section adjacent and against cutting blade 212 which thereby severs the tissue section. The dissected tissue section remains grasped by grasper leg 70 and the instrument is removed leaving the free ends of the Fallopian tube ligated together by the loop of suture material.

The ligating instrument 10 is preferably provided as an instrument assembly containing one or more ligation squid assemblies 124 and a reusable transecting squib assembly 200. Thus, a new ligating assembly 124 can be attached to ligation instrument 10 and the opposite Fallopian tube ligated in similar manner.

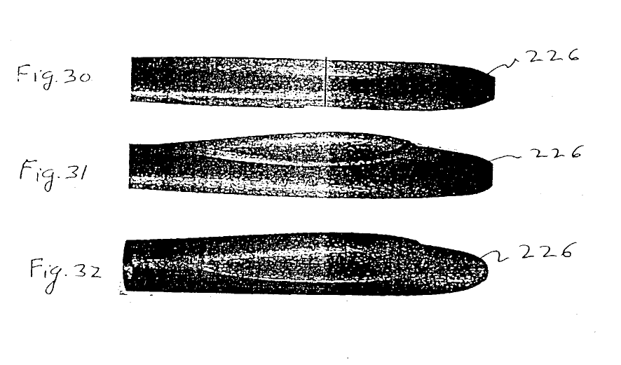

Referring to FIGS. 30-32, there is disclosed an over molded part 226 for use with at least part of handle body 34. Overmold 226 is formed of a relatively resilient material to increase the comfort in holding ligating instrument 10 and provide a better grip on ligating instrument 10. Over mold 226 can be formed of various materials, such as, plastics, foams, etc.

It will be understood that various modifications can be made to the embodiments disclosed herein. For example, other SULU's having different functions may be used with the disclosed one handled instrument having an integral grasper. Therefore, the above description should not be construed as limiting but merely as exemplifications of preferred embodiments. Those skilled in the art will envision other modifications within the scope and spirit of the claims appended hereto.

1. A ligating instrument comprising:
a handle assembly;
an elongate tubular member extending distally from the handle assembly;
an end effector mounted on a distal end of the elongate tubular member and operable to hold open a loop of material;
a grasping member extendable from a distal end of the elongate tubular member; and
a grasp actuator integral with the handle assembly such that actuation of the grasp actuator moves the grasping member relative to the distal end of the elongate tubular member.

2. The ligating instrument as recited in claim 1, wherein actuation of the grasp actuator extends the grasping member distally of the distal end of the elongate tubular member.

3. The ligating instrument as recited in claim 2, wherein the grasping member is extended through the loop of material held by the end effector.

4. The ligating instrument as recited in claim 1, wherein the end effector is removably mounted to the distal end of the elongate tubular member.

5. The ligating instrument as recited in claim 1, further comprising an end effector actuator, integral with the handle assembly, the end effector actuator operable to hold open the loop of material.

6. The ligating instrument as recited in claim 5, wherein the grasp actuator and the end effector actuator are operable by the same single hand while the handle assembly is held by the single hand.

7. The ligating instrument as recited in claim 1, further comprising an over mold positioned at least partially about the handle assembly.
